(19) United States
(12) Patent Application Publication
(10) Pub. No.: US 2025/0069423 A1
(43) Pub. Date: Feb. 27, 2025

(54) COMPACT WHOLE SLIDE IMAGE REPRESENTATION LEARNING WITHOUT MEMORY BOTTLENECK

(71) Applicant: Mayo Foundation for Medical Education and Research, Rochester, MN (US)

(72) Inventors: Hamid R. Tizhoosh, Rochester, MN (US); Sobhan Hemati, Waterloo (CA)

(21) Appl. No.: 18/815,717

(22) Filed: Aug. 26, 2024

Related U.S. Application Data
(60) Provisional application No. 63/578,629, filed on Aug. 24, 2023.

(51) Int. Cl. G06V 20/69; G06T 7/00; G16H 50/20

(52) U.S. Cl. CPC G06V 20/698; G06T 7/0012; G16H 50/20; G06T 2207/30096

(57) ABSTRACT

Compact whole slide image (WSI) representations can be learned using a suitably trained generative model. The generative model is trained on training data using an instance-based training. Using gradient sparsity and quantization losses, the generative model learns to generate compact (e.g., sparse and binary) representations and/or embeddings of whole slide images.

### CROSS-REFERENCE TO RELATED APPLICATIONS

This application claims the benefit of U.S. Provisional Patent Application Ser. No. 63/578,629, filed on Aug. 24, 2023, and entitled “COMPACT WHOLE SLIDE IMAGE REPRESENTATION LEARNING WITHOUT MEMORY BOTTLENECK,” which is herein incorporated by reference in its entirety.

### BACKGROUND

The widespread adoption of digital pathology has spurred the digitization of tissue biopsy samples, known as whole slide images (WSIs). The computational pathology is expected to reduce physician workload, improve diagnostic performance, and facilitate teaching and research in pathology.

Deep learning is a successful tool for image analysis, including various applications in the medical domain. However, deep networks are challenging to adapt for WSI analysis. These challenges include, but are not limited to, tissue textures, rotationally invariant nature of the tissue, staining variations, and lack of fine-grained (i.e., patch-level) labeled data. Among all challenges, the major challenge is the sheer size of WSIs, typically 50,000 by 50,000 pixels. Furthermore, WSIs are arranged in a multi-resolution pyramidal structure containing images at different magnifications. Therefore, memory and computationally efficient frameworks for WSI analyzing are an urgent need.

Patch extraction is typically the first step for the representation learning of a WSI. Extracting thousands of representative patches from a WSI, the representation of WSIs for classification and retrieval systems is a non-trivial task. Processing the patches separately instead of the entire WSI eases the memory bottleneck; however, this leads to multi-vector embedding, which is non-trivial to transform to a single vector representation introducing new challenges, such as high data usage and compromised retrieval speed. Considering this, computing a single-vector representation of a WSI is an active area of research. Ideally, we are interested in a deep-learning solution that can be efficiently trained on WSI patches (at various magnifications), yielding a compact single-vector representation for the WSI, much more suitable for efficient retrieval tasks.

Multiple instance learning (MIL) enables learning on set data instead of using single instances during training. Although MIL has become a preferred method for WSI representation, it does have several limitations. Among others, MIL requires all instances to be processed at once as a set (e.g., a bag), making it difficult to develop end-to-end training in a memory-efficient manner. Another issue with existing WSI representation methods is that the obtained embeddings cannot be directly used for WSI search in its raw form. Searching within large archives of WSIs through the nearest neighbor search leads to a prohibitively large increase in memory demand and retrieval times. As a result, an ancillary processing method is usually necessary to encode these embeddings into more suitable forms, such as binary and sparse embeddings facilitating the speed and memory efficiency in nearest neighbor search.

Finally, current WSI engines (e.g., Yottixel, SMILY) ignore the a priori knowledge, such as tumor type, about WSIs for performing the search. It would be advantageous to employ all known attributes of WSIs for producing a more effective embedding.

### SUMMARY OF THE DISCLOSURE

It is an aspect of the present disclosure to provide a method for generating representation data of whole slide image data. The method includes accessing whole slide image (WSI) data with a computer system. A machine learning model is also accessed with the computer system, where the machine learning model includes a generative model that has been trained on training data to generate WSI embeddings from whole slide images. The WSI data are input to the machine learning model, generating WSI representation data as an output. The WSI representation data include at least one of WSI embeddings or classifications for the WSI data. The WSI representation data may then be output via the computer system.

It is another aspect of the present disclosure to provide a method for training a generative model to generate whole slide image embeddings. Training data are accessed with a computer system, where the training data include at least one of whole slide images or whole slide image patches. Using the computer system, a generative model is trained on the training data based on a gradient sparsity loss and a gradient quantization loss. In this way, the generative model is trained to generate compact whole slide image embeddings. The trained generative model is then stored with the computer system.

### BRIEF DESCRIPTION OF THE DRAWINGS

FIG. 1A shows an example generative model architecture and associated instance-based training scheme for learning compact whole slide image representations, embeddings, and/or classifications.

FIG. 1B shows an example of a workflow for using the trained generative model for obtaining WSI embeddings for a set of patches of a whole slide image.

FIG. 2 is a flowchart setting forth the steps of an example method for generating WSI representation data from WSI data using a suitably trained neural network or other generative model.

FIG. 3 is a flowchart setting forth the steps of an example method for training a neural network or other generative model to generate compact WSI embeddings from WSI data.

FIG. 4 shows an example of feature values across the first 5,000 high variance dimensions for a whole slide image using the C-Deep-SFV and C-Deep-FV techniques described in the present disclosure.

FIG. 5 shows an example of gradient sparsity loss ( [symbol] ₁ norm of the loss function gradient) of C-Deep-SFV and C-Deep-FV during training epochs.

FIG. 6 is a block diagram of an example WSI image classification and retrieval system that can implement the methods described in the present disclosure.

FIG. 7 is a block diagram of example components that can implement the system of FIG. 6.

### DETAILED DESCRIPTION

Described here are systems and methods for learning compact whole slide image (WSI) representations. The disclosed framework is based on deep conditional generative modeling and Fisher vector theory. Unlike the common practice to represent WSIs (i.e., patch-oriented MIL schemes), the training for the disclosed systems and methods is instance-based. As a result, memory usage (e.g., GPU memory usage) is significantly reduced. Advantageously, the disclosed systems and methods may be trained end-to-end by feeding individual instances instead of a bag of instances, which reduces time and memory bottlenecks, thereby enabling the systems and methods to incorporate patches at multiple magnification levels without running into memory issues.

As will be described below in more detail, two loss functions may be used for learning sparse and binary permutation-invariant representations for WSIs. In some implementations, the obtained sparse and binary WSI embeddings may be referred to as Conditioned Sparse Fisher Vector (C-Deep-SFV) and Conditioned Binary Fisher Vector (C-Deep-BFV) embeddings. These loss functions, which may be based on gradient sparsity and gradient quantization for learning sparse and binary permutation-invariant representations are advantageous for efficient WSI retrieval tasks. As an example, the gradient sparsity loss function forces the generative model to use parameters for generating a sample, and as a result, the dimensionality of the WSI embeddings can be reduced while still achieving good performance.

The systems and methods described in the present disclosure implement a new type of Fisher Vector based on deep generative models, such as variational autoencoders (VAEs), for WSI representation learning. Advantageously, by using generative models (e.g., VAEs) for WSI representation learning, higher-order statistics can be captured while learning a set representation. This is an improvement over previous Gaussian mixture model (GMM) based Fisher Vector techniques, which capture no more than second-order statistics of data for set encoding. Additionally, the disclosed systems and methods can be adapted to include a classification loss to the training such that the available WSI level primary diagnosis labels may be employed during training of the VAE. As yet another advantage, the generative models (e.g., VAEs) can be designed to be conditioned on available information, such as the tumor type. Given the fact that every tumor type has its own specific cancer subtypes, this conditioning improves the quality of the resultant WSI embeddings. In this way, representation learning can be guided by a priori information (e.g., the tumor type) as a way of self-supervision. As noted above, the systems and methods described in the present disclosure also utilize two novel loss functions for compact (sparse and binary) and permutation-invariant WSI representation learning. These compact and permutation-invariant WSI representations are advantageous for efficient WSI searching in large archives.

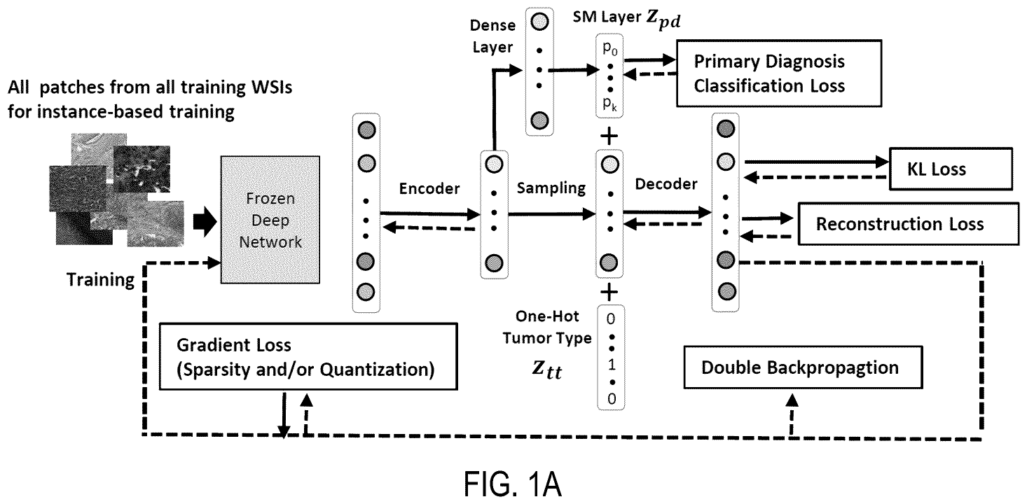

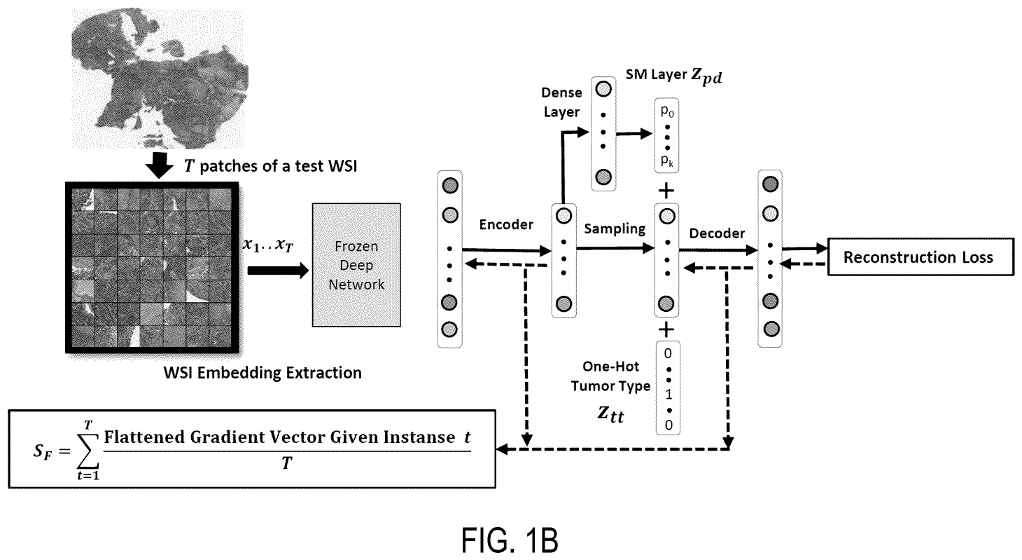

FIG. 1A illustrates an example machine learning model architecture that can be implemented in the systems and methods described in the present disclosure, and FIG. 1B illustrates an example procedure for obtaining WSI embedding from patches. The example architecture shown in FIG. 1A is a conditioned VAE (i.e., conditioned on tumor type in this example) with Kullback-Leibler (KL) and reconstruction losses, and which also has a classification loss for primary diagnosis. As a non-limiting example, a frozen pretrained convolutional neural network (e.g., DenseNet-121) may be used as the backbone of the VAE. In this example, each encoder and decoder parts contain three fully connected layers. The last layer of the encoder is fed to a softmax layer (SM Layer in FIG. 1A) for primary diagnosis prediction. In order to condition the VAE, for each patch, the output of the softmax layer along with a one-hot encoded vector representing the available tumor type information of the patch is concatenated to the latent vector. Then, this vector is fed to the decoder part. As it can be seen from FIG. 1A, the CVAE is trained on a per-instance basis enabling it to even include patches from multiple magnifications. Further, to obtain compact WSI embedding, the gradient can be regularized to have sparse or minimum quantization loss gradients. After training the network, Fisher Vector Theory can be implemented to obtain a Fisher Score (S_{f}). The Fisher Vector, which is the WSI embedding, can subsequently be extracted.

Described now is an example framework for learning compact WSI representations. The proposed method is memory efficient during training and learns representations that are permutation-invariant, compact (sparse/binary), and can be conditioned on known information (e.g., the given tumor type) for self-supervision. The disclosed systems and methods can be trained in an end-to-end manner on individual instances instead of a bag of instances to obtain representations for both patches and the WSI in its entirety.

When implementing a Fisher kernel, the kernel function can be derived from a generative probability model. To take advantage of generative models in discriminative tasks, the gradient space of the generative models can be employed to use the generative process as a similarity metric between examples (or set of examples, i.e., X={xₜ, t=1, . . . , T} where T is the number of examples in the set). Consider a class of probability models p(X|θ) where θ∈θ is a parameter vector and X is set of examples, i.e., X={xₜ, t=1, . . . , T}. The Fisher Score may then be defined as

$${U}_{X} = {∇}_{θ} log p ( X \vert θ ) ; \qquad ( 1 )$$

where U_{X}∈ [symbol] . The dimensionality, d, of the Fisher Score is equal to the number [symbol] of parameters in the generative model p(X|θ) independent of the number of data points in the set T. The Fischer Information Matrix (FIM) is:

$$I = {E}_{x \sim p ( x \vert θ )} \{ {U}_{X} {U}_{X}^{T} \} . \qquad ( 2 )$$

Subsequently, the Fisher kernel can be defined as:

$$K ( X , Y ) = {U}_{X}^{T} {I}^{- 1} {U}_{Y} . \qquad ( 3 )$$

The Fisher kernel can be used to calculate the similarity between two sets of data points. A GMM-based Fisher vector may be used as a way to encode a set of local descriptors in a single embedding, where the Fisher vector is the normalized Fisher score, s_{F}, which may be calculated as:

$${s}_{F} = \frac{1}{T} L {∇}_{θ} log p ( X \vert θ ) = L \frac{1}{T} {\sum }_{t = 1}^{T} {∇}_{θ} log p ( {x}_{t} \vert θ ) ; \qquad ( 4 )$$

where L can be calculated from the Cholesky decomposition of the inverse FIM; that is, I⁻¹=L^{T}L, with the assumption that data points in X are statistically independent.

As described above, although GMM-based Fisher vectors captures second-order statistics for obtaining the set representation, there are some issues that makes them unsuitable for WSI representation learning. First, GMMs are trained in a fully unsupervised manner while considering the challenges inherent to pathology images (e.g., challenging textures, color variations, etc.) employing available information; that is, primary site or primary diagnosis of the WSI is needed to obtain a compact global representation. Second, GMMs are sub-optimal because they cannot be applied in an end-to-end manner to the images. GMMs are also not able to fully capture the natural clustering of patch descriptors due to the inefficient training scheme used in GMMs and the fact that by employing the GMMs, no more than second-order statistics of data are captured using Fisher vectors. The systems and methods described in the present disclosure overcome these limitations by providing a new type of Fisher Vector based on deep generative models for WSI representation learning.

A VAE is first trained and then modified to be conditioned on tumor type. As noted above, a classification loss can be added to the end of the encoder part of the VAE such that primary diagnosis label information can be injected into the model space. As also noted above, two novel loss functions may be used for learning sparse and binary permutation-invariant representations.

To learn the encoder and decoder parameters of the VAE, i.e., ϕ and θ, that models distribution of x, the prior distribution on the random variable z can be assumed as p_{θ}(z) and as a result, xₜ is sampled from p_{θ}(x|z). In this case, one can show that the lower bound for the log p_{θ}(x) can be calculated as:

$$log {p}_{θ} ( x ) \geq - {q}_{ϕ} ( z \vert x ) {p}_{θ} ( z ) + {E}_{{q}_{ϕ} ( z \vert x )} [ log {p}_{θ} ( x \vert z ) ] ; \qquad ( 5 )$$

where q_{ϕ}(z|x) is the approximate posterior with parameters ϕ. The lower bound in Eqn. (5) is known as a variational lower bound and on the patch xₜ and it is represented with [symbol] (ϕ, θ, xₜ). This lower bound can be maximized to learn the generative model parameters. In the context of the VAE model, q_{ϕ}(zₜ|xₜ) and p_{θ}(xₜ|zₜ) are the encoder and decoder, respectively. In order to learn the encoder and decoder parameters (i.e., ϕ and θ), the prior distribution p_{θ}(zₜ) is [symbol] (zₜ; 0,I) and q_{ϕ}(zₜ|xₜ) and p_{θ}(xₜ|zₜ) follow the normal distributions [symbol] (zₜ; μ_{z}_{ₜ}, σ_{z}_{ₜ}²I) and [symbol] (xₜ; μₓ_{ₜ}, σₓ_{ₜ}²I). In this case, by estimating the latent code using one-step Monte Carlo, the variational lower bound is:

$$ℒℬ ( ϕ , θ , {x}_{t} ) = log {p}_{θ} ( {x}_{t} \vert {z}_{t} ) + \frac{1}{2} {\sum }_{j = 1}^{d} ( 1 + log {σ}_{{z}_{t} ( j )}^{2} ) - \frac{1}{2} {‖ {μ}_{{z}_{t}} ‖}^{2} - \frac{1}{2} {‖ {σ}_{{z}_{t}} ‖}^{2} ; \qquad ( 6 )$$

where zₜ is sampled from [symbol] (xₜ; μₓ_{ₜ}, σₓ_{ₜ}²I).

When the tumor type of a WSI is available, the VAE may be conditioned on the tumor type of the given WSI to draw benefit from this a priori knowledge. Tumor type may be represented as a one-hot encoded vector zₜₜ, which can be concatenated to zₜ. Furthermore, to inject WSI-level primary diagnosis information into the generative model, a classification loss can be added to the last layer of the encoder before the sampling layer. The WSI label can be assigned to all patches extracted from that WSI. Then, the softmax of predicted primary diagnosis z_{pd}, with length k, can be concatenated to the latent space. The latent space associated with xₜ that is fed to decoder can be modified as zₜ←[zₜ, zₜₜ, z_{pd}]. Considering the classification loss, so far, the loss function for training the Conditioned Variational Autoencoder (CVAE) has the form:

$${ℒ}_{CVAE} = {λ}_{1} {ℒ}_{ref} + {λ}_{2} {ℒ}_{kl} + {λ}_{3} {ℒ}_{cls} ; \qquad ( 7 )$$

where [symbol] _{rec}, [symbol] ₖₗ, [symbol] _{cls} are reconstruction, Kullback-Leibler divergence, and classification losses. Minimizing the first two terms is equivalent to maximizing the variational lower bound, and the third loss is the classification loss of predicting cancer subtypes.

A method for learning a Conditioned Deep Sparse Fisher Vector (C-Deep-SFV) is now described. As the gradient space represents the WSI, sparsity in the gradient can be encouraged by adding the [symbol] ₁ norm of the gradient of the loss function in Eqn. (7) to the overall training loss. To regularize the gradient, a double backpropagation can be utilized, where given a batch of data points X, the loss function can be written as:

$${ℒ}_{SFV} = {ℒ}_{CVAE} + {λ}_{4} {\sum }_{{W}_{i} ∈ 𝕎} {‖ {∇}_{{W}_{i}} {ℒ}_{CVAE} ( 𝕎 , X ) ‖}_{1} ; \qquad ( 8 )$$

where [symbol] is the set of CVAE parameters for all layers, Wᵢ and ∇_{W}_{ᵢ} [symbol] _{CVAE}( [symbol] , X) are the is parameters and the gradient of the CVAE loss with respect to the i^{th} layer parameters.

Advantageously, using a gradient sparsity loss contributes to the compactness of WSI embeddings, which leads to high efficiency for WSI search in terms of memory usage and retrieval times. After encouraging sparsity on the gradients, the C-Deep-SFV can represent a WSI by significantly fewer parameters leading to compact representations. As another advantage, models trained using this gradient sparsity-based loss function achieve better generalization. For instance, increasing the sparsity in gradients may lead to many zero values for gradients. Considering the learning rule for updating the network parameters, zero gradients lead to no updates in some specific weights. This can be seen as the network learning not to learn some patterns in data (e.g., the noise), which may reduce overfitting. Encouraging sparsity in gradients also leads to many zero values in gradients given a batch of data. One can see this as a dropout-like operation, such as a gradient dropout. Encouraging sparsity in gradients can also avoid over training. From the Fisher kernel theory perspective, when gradients with respect to more parameters are zero, this means that the generative model is using a smaller number of parameters to generate samples. As a result, when sparsity in the gradients is encouraged, the neural network is forced to use a smaller portion of its power to generate samples.

For learning Conditioned Deep Binary Fisher Vector (C-Deep-BFV), inspired by the quantization-based learning in hashing literature, the quantization loss of the gradient of the CVAE loss can be reduced with respect to the parameters of each layer. As an example, the following can be found:

$${arg min}_{{B}_{i} , {∇}_{{W}_{i}} {ℒ}_{CVAE} ( 𝕎 , X )} {\sum }_{{W}_{i} ∈ 𝕎} {‖ {∇}_{{W}_{i}} {ℒ}_{CVAE} ( 𝕎 , X ) {B}_{i} ‖}_{2}^{2} s . t . {B}_{i} ∈ {\{ - 1 , 1 \}}^{{d}_{i} \times 1} ; \qquad ( 9 )$$

where Bᵢ is the flattened binary representation of the gradient of the CVAE loss with respect to parameters of the ith layer. In this case, the loss function to obtain BFV can be written as:

$${ℒ}_{BFV} = {ℒ}_{CVAE} + {λ}_{5} {\sum }_{{W}_{i} ∈ 𝕎 , {B}_{i}} {‖ {∇}_{{W}_{i}} {ℒ}_{CVAE} ( 𝕎 , X ) - {B}_{i} ‖}_{2}^{2} ; \qquad ( 10 )$$

where [symbol] is the set of closest Hamming vertices to gradients with respect to all [symbol] layers. Given the binary optimization variable, Bᵢ, on each epoch, a coordinate descent approach can be employed and each of Bᵢ and Wᵢ can be updated while the other is fixed. For the case that Wᵢ is fixed the problem turns to:

$${arg max}_{{B}_{i}} {‖ {∇}_{{W}_{i}} {ℒ}_{CVAE} ( 𝕎 , X ) - {B}_{i} ‖}_{2}^{2} s . t . {B}_{i} ∈ {\{ - 1 , 1 \}}^{{d}_{i} \times 1} ; \qquad ( 11 )$$

where by expanding Eqn. (11) the above minimization is equivalent to:

$${arg max}_{{B}_{i}} ( {B}_{i}^{T} \cdot {∇}_{{W}_{i}} {ℒ}_{CVAE} ( 𝕎 , X ) ) s . t . {B}_{i} ∈ {\{ - 1 , 1 \}}^{{d}_{i} \times 1} . \qquad ( 12 )$$

This problem has the following closed-form solution:

$${B}_{i} = sgn ( {∇}_{{W}_{i}} {ℒ}_{CVAE} ( 𝕎 , X ) ) . \qquad ( 13 )$$

The loss function can be for fixed Bᵢ as:

$${ℒ}_{BFV} = {ℒ}_{CVAE} + {λ}_{5} {\sum }_{{W}_{i} ∈ 𝕎} {‖ {∇}_{{W}_{i}} {ℒ}_{CVAE} ( 𝕎 , X ) - {B}_{i} ‖}_{2}^{2} . \qquad ( 14 )$$

This is similar to SFV learning in Eqn. (8). The variables can be updated using double backpropagation.

Knowing that the length of obtained WSI embeddings is equal to the number of parameters in the generative model, compact (e.g., short) binary codes may be used for more efficient WSI retrieval. Both the gradient sparsity and gradient quantization losses described above can be used to achieve Conditioned Deep Sparse Binary Fisher Vector (C-Deep-BFV). Gradient sparsity pushes the generative model to use fewer parameters to generate a data point. As a result, the quality of embedding will be more robust to dropping some dimensions (e.g., gradient with respect to some parameters of VAE). To choose effective dimensions for each tumor type, the top M parameters that provide the highest variance in their respective gradient values for the training data can be selected.

After the training phase, to obtain a single embedding for a WSI, all patches of that WSI are fed to the CVAE, as shown in FIG. 1B. Then, given the reconstruction loss, the average gradient over all patches is calculated using backpropagation to obtain the Fisher score (s_{F}). Based on Fisher theory, L is also obtained from the FIM to normalize the vector and derive the Fisher vector. Given the computational load of calculating L, it may be replaced with an identity matrix and the gradient may be normalized using power and [symbol] ₂ normalization steps. In other words, representing the power and [symbol] ₂ normalization steps as an operator, [symbol] (⋅), the conditioned deep compact Fisher vector v_{F} can be calculated from the Fisher score s_{F} as:

$${v}_{F} = ( \frac{1}{T} {\sum }_{t = 1}^{T} {∇}_{θ , ϕ} {‖ {x}_{t} - {\hat{x}}_{t} ( θ , ϕ ) ‖}_{2}^{2} ) = ( {s}_{F} ) ; \qquad ( 15 )$$

Where xₜ and \hat{x}ₜ(θ, ϕ) are the patch embedding and its reconstruction, respectively. The size of the proposed feature vector is equal to the number of parameters in CVAE. The test-time, the one-hot vector of the tumor type, can be fed to the CVAE as a known parameter while the z_{pd} may be calculated by the classifier. For cases in which there are multiple WSIs per patient, all patches from all WSIs of the patient can be fed to the generative model, the gradients can be calculated and then averaged, and one embedding per patient can be obtained.

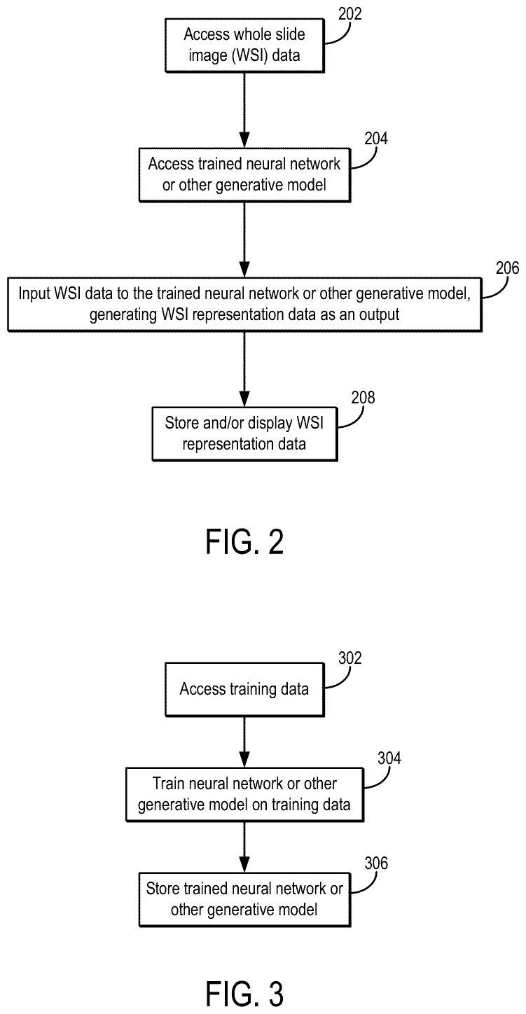

Referring now to FIG. 2, a flowchart is illustrated as setting forth the steps of an example method for generating representations of whole slide images using a suitably trained neural network or other machine learning algorithm. As will be described, the neural network or other machine learning algorithm takes WSI data as input data and generates WSI representation data as output data. As an example, the WSI representation data can include representations of the input WSI data at varying levels of magnification, such as an entire whole slide image, individual patches of a whole slide image, different levels of magnification of a whole slide image or patches thereof, and so on.

The method includes accessing WSI data with a computer system, as indicated at step 202. Accessing the WSI data may include retrieving such data from a memory or other suitable data storage device or medium. Additionally or alternatively, accessing the WSI data may include acquiring such data and transferring or otherwise communicating the data to the computer system. For example, WSI data can be acquired with a slide scanner or other suitable imaging system. In some instances, the WSI data may include whole slide images that depict a histopathology sample (e.g., cell sample(s), tissue sample(s)). In other instances, the WSI data may include preprocessed whole slide images, such as whole slide images that have been divided into tiles or patches.

A trained neural network (or other suitable machine learning algorithm) is then accessed with the computer system, as indicated at step 204. In general, the neural network is trained, or has been trained, on training data in order to generate representations of the input whole slide images, image patches, or both. Additionally or alternatively, the neural network may be trained to generate a classification of the WSI data and/or to generate embeddings of each whole slide image, individual image patches, or both.

Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed.

An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs.

The WSI data are then input to the one or more trained neural networks, generating output as WSI representation data, as indicated at step 206. For example, the WSI representation data may include representations of the input WSI data at varying levels of magnification, such as an entire whole slide image, individual patches of a whole slide image, different levels of magnification of a whole slide image or patches thereof, and so on. Additionally or alternatively, the WSI representation data may include a classification of whole slide images, images patches, or both. For instance, the WSI representation data can include a classification of the WSI data indicating a particular disease type, disease severity, and so on. In some other instances, the WSI representation data may include embeddings for whole slide image and/or image patches in the WSI data.

In some instances, the WSI representation data may include an average of the gradients calculated over all of the image patches in the WSI data. In these instances, a Fisher score may be computed from these average values, as described above, and stored as part of the WSI representation data. Additionally or alternatively, a Fisher vector may be calculated as described above and stored as part of the WSI representation data.

The WSI representation data generated by inputting the WSI data to the trained neural network(s) can then be displayed to a user, stored for later use or further processing, or both, as indicated at step 208. For example, the WSI representation data may be used to provide WSI search and retrieval with reduced memory burden by more efficiently representing and cataloging the whole slide images and/or image patches in the WSI data. Additionally or alternatively, the WSI representation data may be presented to a user. For instance, the WSI representation data may include classifications of whole slide images and/or images patches contained in the WSI data. In these instances, the classification(s) may be presented to the user along with the corresponding WSI data.

Referring now to FIG. 3, a flowchart is illustrated as setting forth the steps of an example method for training one or more neural networks (or other suitable machine learning algorithms) on training data, such that the one or more neural networks are trained to receive WSI data as input data in order to generate WSI representation data as output data.

In general, the neural network(s) or other machine learning models are generative models. When the generative model is a neural network, it may implement any number of different neural network architectures. For instance, the neural network(s) could implement a convolutional neural network, a residual neural network, or the like. As one non-limiting example, the neural network may implement a variational autoencoder model, which may be a conditioned variational autoencoder model as described above. Alternatively, the neural network(s) could be replaced with other suitable generative models, such as those based on supervised learning, unsupervised learning, deep learning, ensemble learning, dimensionality reduction, and so on.

The method includes accessing training data with a computer system, as indicated at step 302. Accessing the training data may include retrieving such data from a memory or other suitable data storage device or medium. Alternatively, accessing the training data may include acquiring such data with a slide scanner or other suitable imaging system.

In general, the training data can include whole slide images and/or image patches extracted from whole slide images. In some instances, the training data may include whole slide images and/or image patches at various levels of magnification. Additionally, the training data may include other data, such as classification data (e.g., disease type, tumor type, disease severity, etc.). In some embodiments, the training data may include whole slide images and/or image patches that have been labeled (e.g., labeled as containing patterns, features, or characteristics indicative of particular disease types, states, and/or severities; and the like).

The method can include assembling training data from whole slide images and/or image patches using a computer system. This step may include assembling the whole slide images and/or image patches into an appropriate data structure on which the neural network or other machine learning algorithm can be trained. Assembling the training data may include assembling whole slide images and/or image patches and other relevant data. For instance, assembling the training data may include generating labeled data and including the labeled data in the training data. Labeled data may include whole slide images and/or image patches or other relevant data that have been labeled as belonging to, or otherwise being associated with, one or more different classifications or categories.

One or more neural networks (or other suitable machine learning algorithms) are trained on the training data, as indicated at step 304. In general, the neural network can be trained by optimizing network parameters (e.g., weights, biases, or both) based on minimizing one or more loss functions. As described above, the neural network(s) may be trained while minimizing sparse and/or binary gradient losses. Additionally or alternatively, the neural network(s) may be trained while minimizing additional losses, such as primary diagnosis loss, KL loss, and reconstruction loss.

Training a neural network may include initializing the neural network, such as by computing, estimating, or otherwise selecting initial network parameters (e.g., weights, biases, or both). During training, an artificial neural network receives the inputs for a training example and generates an output using the bias for each node, and the connections between each node and the corresponding weights. For instance, training data can be input to the initialized neural network, generating output as WSI representation data. The artificial neural network then compares the generated output with the actual output of the training example in order to evaluate the quality of the WSI representation data. For instance, the WSI representation data can be passed to one or more loss functions to compute one or more errors. The current neural network can then be updated based on the calculated error (e.g., using backpropagation, double backpropagation, etc.). For instance, the current neural network can be updated by updating the network parameters (e.g., weights, biases, or both) in order to minimize the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current neural network and its associated network parameters represent the trained neural network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The artificial neural network can be constructed or otherwise trained based on training data using one or more different learning techniques, such as supervised learning, unsupervised learning, reinforcement learning, ensemble learning, active learning, transfer learning, or other suitable learning techniques for neural networks. As an example, supervised learning involves presenting a computer system with example inputs and their actual outputs (e.g., categorizations). In these instances, the artificial neural network is configured to learn a general rule or model that maps the inputs to the outputs based on the provided example input-output pairs.

The one or more trained neural networks are then stored for later use, as indicated at step 306. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

As an example, the systems and methods described in the present disclosure were used to generate WSI representation data including WSI embeddings for both search and classification tasks. The datasets employed for this example study included diagnostic slides from The Cancer Genomic Atlas (TCGA) repository and the Liver-Kidney-Stomach (LKS) immunofluorescence.

For this experiment, 40% of the TCGA diagnostic WSIs were randomly selected as a test set and the rest for training. For both test and training WSIs, 15% of patches with 1000×1000 patch size were selected based on the Yottixel (the same clustering method has been applied). The vertical search was applied on the test set (3,761 WSIs), and leave-one-out patient performed for searching WSIs through the same primary site. The majority of the top 3 similar cases were used for predicting each query cancer subtype. Yottixel takes the median of minimum patch distances to calculate two WSIs dissimilarity, while C-GMM-FV and the proposed method obtain one embedding per WSI.

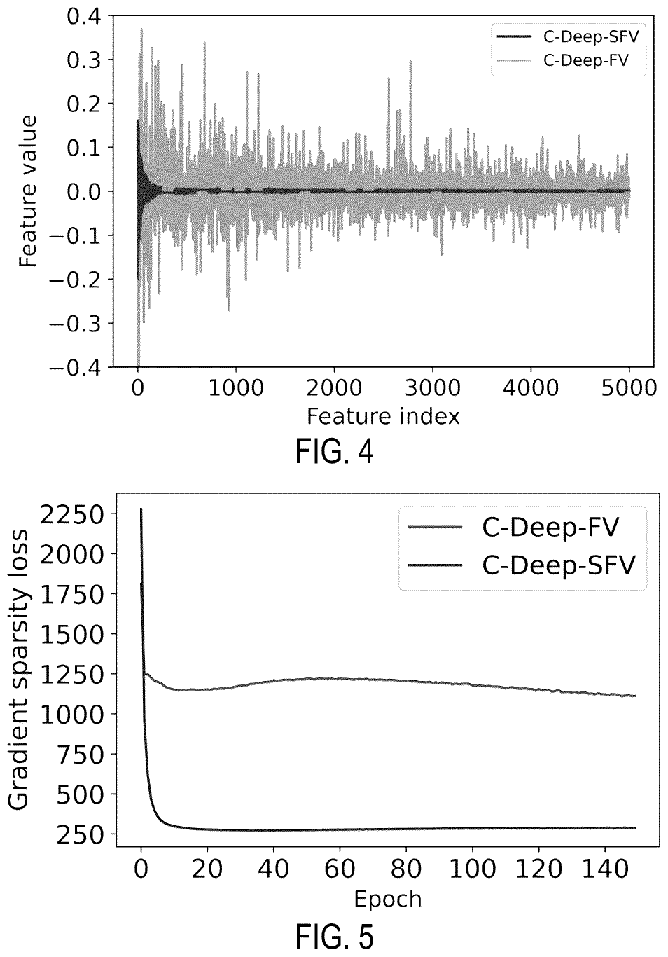

The disclosed systems and methods improved the search F1-measure for all 29 cancer subtypes while the embeddings were binary and/or sparse. Although in almost all subtypes of two primary sites (Gynecological and Prostate/testis), C-Deep-FV performed better than other methods, almost in all cases, compact WSI embeddings obtained by gradient sparsity and quantization losses achieved even better search performance. The compactness of the proposed embeddings leads to high efficiency for WSI search in terms of memory usage and retrieval times. FIG. 4 shows the embedding for C-Deep-FV and C-Deep-SFV across the first 5,000 high variance dimensions given the tissue type of the given WSI out of 1,407,105 parameters of our CVAE. Considering FIG. 4, after encouraging sparsity on the gradients, the C-Deep-SFV can represent the WSI by significantly much fewer parameters leading to compact representations. FIG. 5 shows the effectiveness of incorporating gradient sparsity loss in reducing the [symbol] ₁ norm of the loss function gradient during the epochs.

The length of the proposed WSI embedding is equal to the number of trainable parameters of the generative model, which was 1,407,105 in this example. Although a relatively small generative model was employed for models with millions of parameters, the embeddings may not be suitable for efficient WSI search. The proposed gradient sparsity solves this issue by enforcing the generative model to use a smaller number of parameters for generating samples. In other words, by imposing sparsity on gradients, the parameters that the gradients are zero with respect to them do not have a significant contribution to generating those samples, so they can be removed from embeddings.

The effect of the sparsity loss was validated by selecting the gradients with respect to a subset of some parameters that leads to high-variance gradients per tissue type. Based on the Fisher Vector Theory this is intuitive that the gradients with respect to generative model parameters show the contribution of each parameter to generating a sample. More precisely, by encouraging sparsity on the gradients, fewer parameters are contributing to generation; consequently, more parameters can be dropped in the final embedding.

In another example study, the systems and methods described in the present disclosure were tested for a WSI task. For this task, the quality of obtained WSI embeddings were validated on both TCGA and LKS datasets. For both cases, a simple, fully connected network with two layers on top of the SFV embeddings was trained for the purpose of WSI classification.

Lung Adenocarcinoma (LUAD) and Lung Squamous Cell Carcinoma (LUSC) are two main types of non-small cell lung cancer (NSCLC) that account for 65-70% of all lung cancers. Automated classification of these two main subtypes of NSCLC is a crucial step to building computerized decision support systems. The dataset for this experiment included all lung cancer slides in TCGA (approximately 2 TB of data). In this example, 2,580 WSIs were used from TCGA public repository with 774 WSIs for the test and the rest for training.

The Liver-Kidney-Stomach (LKS) is the other publicly available dataset that was used for validating quality of WSI embeddings. The LKS dataset contains immunofluorescence WSIs. The dataset contains 684 WSIs from four classes Anti-Mitochondrial Antibodies (AMA), Negative (Neg), Vessel-Type Anti-Smooth Muscle Antibodies (SMA-V), and Tubule-Type Anti-Smooth Muscle Antibodies (SMA-T). This dataset contains one low-resolution image and also a set of patches per WSI. C-Deep-SFV was compared against Selective Objective Switch (SOS), Reinforced Dynamic Multi-Scale (RDMS) and three techniques for WSI classification, namely Image-Level, Patch-Level, and Conventional Multi-Scale.

For this experiment, only low-resolution images were employed for training the backbone and then for each WSI a low-resolution image was used along with 5% of high-resolution patches for training the CVAE and extracting the WSI embedding. Embeddings obtained by the disclosed systems and methods were compact and suitable for WSI search.

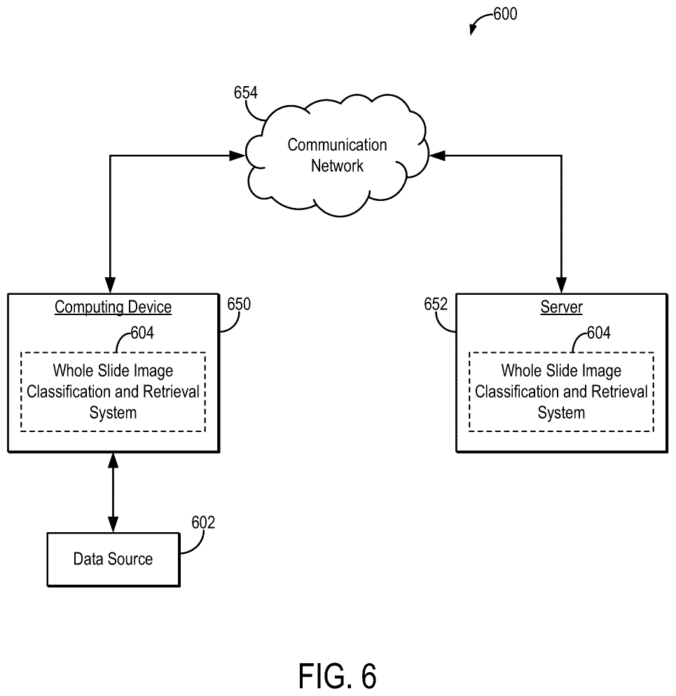

FIG. 6 shows an example of a system 600 for learning or otherwise generating compact representation of WSIs in accordance with some embodiments of the systems and methods described in the present disclosure. As shown in FIG. 6, a computing device 650 can receive one or more types of data (e.g., WSI data) from data source 602. In some embodiments, computing device 650 can execute at least a portion of a whole slide image classification and retrieval system 604 to learn or otherwise generate compact representations of whole slide images from WSI data received from the data source 602.

Additionally or alternatively, in some embodiments, the computing device 650 can communicate information about data received from the data source 602 to a server 652 over a communication network 654, which can execute at least a portion of the whole slide image classification and retrieval system 604. In such embodiments, the server 652 can return information to the computing device 650 (and/or any other suitable computing device) indicative of an output of the whole slide image classification and retrieval system 604.

In some embodiments, computing device 650 and/or server 652 can be any suitable computing device or combination of devices, such as a desktop computer, a laptop computer, a smartphone, a tablet computer, a wearable computer, a server computer, a virtual machine being executed by a physical computing device, and so on. The computing device 650 and/or server 652 can also reconstruct images from the data.

In some embodiments, data source 602 can be any suitable source of data (e.g., whole slide images, patches generated from whole slide images, etc.), another computing device (e.g., a server storing whole slide images, patches generated from whole slide images, etc.), and so on. In some embodiments, data source 602 can be local to computing device 650. For example, data source 602 can be incorporated with computing device 650 (e.g., computing device 650 can be configured as part of a device for measuring, recording, estimating, acquiring, or otherwise collecting or storing data). As another example, data source 602 can be connected to computing device 650 by a cable, a direct wireless link, and so on. Additionally or alternatively, in some embodiments, data source 602 can be located locally and/or remotely from computing device 650, and can communicate data to computing device 650 (and/or server 652) via a communication network (e.g., communication network 654).

In some embodiments, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 654 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, and so on.

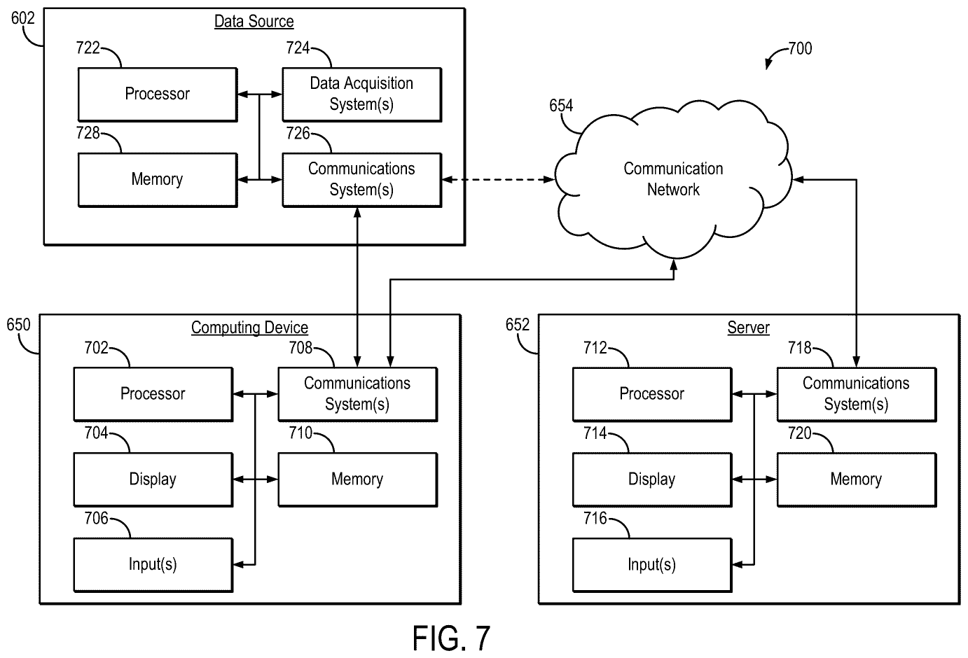

Referring now to FIG. 7, an example of hardware 700 that can be used to implement data source 602, computing device 650, and server 652 in accordance with some embodiments of the systems and methods described in the present disclosure is shown.

As shown in FIG. 7, in some embodiments, computing device 650 can include a processor 702, a display 704, one or more inputs 706, one or more communication systems 708, and/or memory 710. In some embodiments, processor 702 can be any suitable hardware processor or combination of processors, such as a central processing unit (“CPU”), a graphics processing unit (“GPU”), and so on. In some embodiments, display 704 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 706 can include any suitable input devices and/or sensors that can be used to receive user input, such as a keyboard, a mouse, a touchscreen, a microphone, and so on.

In some embodiments, communications systems 708 can include any suitable hardware, firmware, and/or software for communicating information over communication network 654 and/or any other suitable communication networks. For example, communications systems 708 can include one or more transceivers, one or more communication chips and/or chip sets, and so on. In a more particular example, communications systems 708 can include hardware, firmware, and/or software that can be used to establish a Wi-Fi connection, a Bluetooth connection, a cellular connection, an Ethernet connection, and so on.

In some embodiments, memory 710 can include any suitable storage device or devices that can be used to store instructions, values, data, or the like, that can be used, for example, by processor 702 to present content using display 704, to communicate with server 652 via communications system(s) 708, and so on. Memory 710 can include any suitable volatile memory, non-volatile memory, storage, or any suitable combination thereof. For example, memory 710 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 710 can have encoded thereon, or otherwise stored therein, a computer program for controlling operation of computing device 650. In such embodiments, processor 702 can execute at least a portion of the computer program to present content (e.g., images, user interfaces, graphics, tables), receive content from server 652, transmit information to server 652, and so on. For example, the processor 702 and the memory 710 can be configured to perform the methods described herein (e.g., the neural network implementations shown in FIGS. 1A and 1B, the method of FIG. 2, the method of FIG. 3).

In some embodiments, server 652 can include a processor 712, a display 714, one or more inputs 716, one or more communications systems 718, and/or memory 720. In some embodiments, processor 712 can be any suitable hardware processor or combination of processors, such as a CPU, a GPU, and so on. In some embodiments, display 714 can include any suitable display devices, such as an LCD screen, LED display, OLED display, electrophoretic display, a computer monitor, a touchscreen, a television, and so on. In some embodiments, inputs 716 can include any suitable input devices and/or sensors that can be used to receive user input, such as a keyboard, a mouse, a touchscreen, a microphone, and so on.

In some embodiments, communications systems 718 can include any suitable hardware, firmware, and/or software for communicating information over communication network 654 and/or any other suitable communication networks. For example, communications systems 718 can include one or more transceivers, one or more communication chips and/or chip sets, and so on. In a more particular example, communications systems 718 can include hardware, firmware, and/or software that can be used to establish a Wi-Fi connection, a Bluetooth connection, a cellular connection, an Ethernet connection, and so on.

In some embodiments, memory 720 can include any suitable storage device or devices that can be used to store instructions, values, data, or the like, that can be used, for example, by processor 712 to present content using display 714, to communicate with one or more computing devices 650, and so on. Memory 720 can include any suitable volatile memory, non-volatile memory, storage, or any suitable combination thereof. For example, memory 720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 720 can have encoded thereon a server program for controlling operation of server 652. In such embodiments, processor 712 can execute at least a portion of the server program to transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone), and so on.

In some embodiments, the server 652 is configured to perform the methods described in the present disclosure. For example, the processor 712 and memory 720 can be configured to perform the methods described herein (e.g., the neural network implementations shown in FIGS. 1A and 1B, the method of FIG. 2, the method of FIG. 3).

In some embodiments, data source 602 can include a processor 722, one or more data acquisition systems 724, one or more communications systems 726, and/or memory 728. In some embodiments, processor 722 can be any suitable hardware processor or combination of processors, such as a CPU, a GPU, and so on. In some embodiments, the one or more data acquisition systems 724 are generally configured to acquire data, images, or both, and can include a slide scanner or other suitable imaging system. Additionally or alternatively, in some embodiments, the one or more data acquisition systems 724 can include any suitable hardware, firmware, and/or software for coupling to and/or controlling operations of a slide scanner or other suitable imaging system. In some embodiments, one or more portions of the data acquisition system(s) 724 can be removable and/or replaceable.

Note that, although not shown, data source 602 can include any suitable inputs and/or outputs. For example, data source 602 can include input devices and/or sensors that can be used to receive user input, such as a keyboard, a mouse, a touchscreen, a microphone, a trackpad, a trackball, and so on. As another example, data source 602 can include any suitable display devices, such as an LCD screen, an LED display, an OLED display, an electrophoretic display, a computer monitor, a touchscreen, a television, etc., one or more speakers, and so on.

In some embodiments, communications systems 726 can include any suitable hardware, firmware, and/or software for communicating information to computing device 650 (and, in some embodiments, over communication network 654 and/or any other suitable communication networks). For example, communications systems 726 can include one or more transceivers, one or more communication chips and/or chip sets, and so on. In a more particular example, communications systems 726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 728 can include any suitable storage device or devices that can be used to store instructions, values, data, or the like, that can be used, for example, by processor 722 to control the one or more data acquisition systems 724, and/or receive data from the one or more data acquisition systems 724; to generate images from data; present content (e.g., data, images, a user interface) using a display; communicate with one or more computing devices 650; and so on. Memory 728 can include any suitable volatile memory, non-volatile memory, storage, or any suitable combination thereof. For example, memory 728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 728 can have encoded thereon, or otherwise stored therein, a program for controlling operation of data source 602. In such embodiments, processor 722 can execute at least a portion of the program to generate images, transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for generating representation data of whole slide image data, the method comprising:
(a) accessing whole slide image (WSI) data with a computer system;
(b) accessing a machine learning model with the computer system, wherein the machine learning model comprises a generative model trained on training data to generate WSI embeddings from whole slide images;
(c) inputting the WSI data to the machine learning model, generating WSI representation data as an output, wherein the WSI representation data comprises at least one of WSI embeddings or classifications for the WSI data; and
(d) outputting the WSI representation data via the computer system.

2. The method of claim 1, wherein the generative model comprises a variational autoencoder model.

3. The method of claim 2, wherein the variational autoencoder model comprises a conditioned variational autoencoder model that is conditioned on a disease type.

4. The method of claim 3, wherein the disease type is represented by a one-hot encoded vector.

5. The method of claim 1, wherein the generative model has been trained on the training data using at least one of a gradient sparsity loss or a gradient quantization loss.

6. The method of claim 1, wherein the WSI representation data comprise compact representations of the WSI data.

7. The method of claim 6, wherein the compact representations of the WSI data comprise sparse and binary representation of the WSI data.

8. The method of claim 1, wherein the WSI representation data comprise a Fisher Vector.

9. The method of claim 8, wherein the Fisher Vector is generated based on gradients of image patch embeddings from the WSI representation data.

10. The method of claim 1, wherein outputting the WSI representation data comprises displaying the WSI representation data to a user via the computer system.

11. The method of claim 1, wherein the WSI representation data comprise classifications for the WSI data and the generative model has been trained on the training data using at least a primary diagnosis classification loss.

12. A method for training a generative model to generate whole slide image embeddings, the method comprising:
(a) accessing training data with a computer system, wherein the training data comprise at least one of whole slide images or whole slide image patches;
(b) training, using the computer system, a generative model on the training data based on a gradient sparsity loss and a gradient quantization loss to train the generative model to generate compact whole slide image embeddings;
(c) storing the trained generative model with the computer system.

13. The method of claim 12, wherein the gradient sparsity loss encourages sparsity in gradients of whole slide image data.

14. The method of claim 12, wherein the gradient quantization loss is determined based on a binary representation of gradients of whole slide image data.

15. The method of claim 12, wherein the generative model is also trained on the training data based on a primary diagnosis classification loss to train the generative model to generate whole slide image classifications.

16. The method of claim 12, wherein the generative model comprises a variational autoencoder model.

17. The method of claim 16, wherein the variational autoencoder model comprises a conditioned variational autoencoder model that is conditioned on a disease type.

18. The method of claim 17, wherein the disease type is a tumor type.

19. The method of claim 17, wherein the conditioned variational autoencoder is conditioned on the disease type using a one-hot encoded vector.

20. The method of claim 12, wherein the generative model is trained on the training data using instance-based training.
